(19) United States
(12) Patent Application Publication
(10) Pub. No.: US 2026/0334766 A1
(43) Pub. Date: Oct. 29, 2026

(54) MEDICAL DEVICE STEERING DEVICES, ASSEMBLIES, AND ASSOCIATED METHODS

(71) Applicant: Boston Scientific Scimed, Inc., Maple Grove, MN (US)

(72) Inventors: Yu Him Michael AU, Framingham, MA (US); Joseph Phillip ALBINA, Hudson, MA (US); Allyn Narcisse JENSRUD, Milford, MA (US); Greg Joseph TRUMBULL, Littleton, MA (US); Andrew James WHITNEY, Douglas, MA (US)

(73) Assignee: Boston Scientific Scimed, Inc., Maple Grove, MN (US)

(21) Appl. No.: 19/657,290

(22) Filed: Apr. 24, 2026

Related U.S. Application Data
(60) Provisional application No. 63/796,534, filed on Apr. 29, 2025.

(51) Int. Cl. A61B 1/005

(52) U.S. Cl. CPC A61B 1/0052; A61B 1/0057

(57) ABSTRACT

A handle of a medical device may include an actuator and a pulley. Actuation of the actuator causes rotation of the pulley. A pair of wires are coupled to the pulley by a crimp. A longitudinal axis of the crimp is approximately perpendicular to an axis of rotation of the pulley.

### CROSS-REFERENCE TO RELATED APPLICATION(S)

This application claims the benefit of priority of U.S. Provisional Patent Application No. 63/796,534, filed on Apr. 29, 2025, the entirety of which is incorporated herein by reference.

### TECHNICAL FIELD

Various aspects of this disclosure relate generally to steering assemblies of medical devices, and related devices, systems, and methods. In particular, the disclosure relates to devices, systems or assemblies, and methods for coupling steering wires to actuators of medical devices This application claims the benefit of priority of U.S. Provisional Patent Application No. 63/750,477, filed on Jan. 28, 2025, the entirety of which is incorporated herein by reference.

### BACKGROUND

Medical devices, such as medical scopes, may include a handle and a shaft. The shaft may terminate in a distal tip. The shaft may be inserted into a body lumen of a subject or into a working channel of a shaft of another medical device, which may, in turn, be inserted into the body lumen of the subject. The handle may include various actuators for controlling the shaft or the distal tip. For example, the shaft may include an articulation portion. The handle may include actuators for controlling the articulation portion. Activation of the actuators of the handle may actively steer the articulation portion, causing the articulation portion to deflect along one or more planes. For example, the actuators of the handle may cause rotation of a pulley. The pulley may be coupled to one or more wires, and the wires may be coupled to the articulation portion. Rotation of the pulley may cause the wires to be moved proximally or distally, thereby articulating the articulation portion.

### SUMMARY

Each of the aspects disclosed herein may include one or more of the features described in connection with any of the other disclosed aspects. Aspects of the disclosure may relate to steering assemblies of medical devices, and associated devices, systems, and methods.

In some aspects, a handle of a medical device may include an actuator and a pulley. Actuation of the actuator causes rotation of the pulley. A pair of wires are coupled to the pulley by a crimp. A longitudinal axis of the crimp is approximately perpendicular to an axis of rotation of the pulley.

The disclosed devices may include any of the following features, alone or in any combination. The actuator is a first actuator, the pulley is a first pulley, the pair of wires is a first pair of wires, the crimp is a first crimp, and the longitudinal axis is a first longitudinal axis. The handle further includes: a second actuator and a second pulley. Actuation of the second actuator causes rotation of the second pulley. A second pair of wires is coupled to the second pulley by a second crimp. A second longitudinal axis of the second crimp is approximately perpendicular to the axis of rotation. The first longitudinal axis and the second longitudinal axis are approximately parallel to one another and are not coaxial. Each wire of the pair of wires is a multi-strand wire. The pulley includes a ramped surface that slopes toward a center of the pulley. In a tensioned configuration of a wire of the pair of wires, the ramped surface urges the tensioned wire toward the center of the pulley. The pulley includes a radially inner portion and a radially outer portion. The radially inner portion is closer to a rotational axis of the pulley. The ramped surface slopes in a direction toward the radially inner portion. The radially outer portion has a flange shape. The ramped surface extends only partially around the pulley. The pulley includes a gap extending from an outer perimeter of the pulley toward an axle of the pulley. The crimp extends across the gap. The pulley includes a recess on either side of the gap for receiving the crimp. The gap is defined by three straight walls and an open outer side. The pulley includes openings such that the wires are on a first axial side of the pulley on a proximal portion of the pulley and a second axial side of the pulley on a distal portion of the pulley. The gap is on the proximal portion of the pulley.

In another aspect, a handle of a medical device includes an actuator and pulley. Actuation of the actuator causes rotation of the pulley. A pair of wires is coupled to the pulley. The pulley includes a radially inner portion and a radially outer portion. The radially outer portion includes a ramped surface that is inclined toward the radially inner portion.

The disclosed devices may include any of the following features, alone or in any combination. The radially outer portion increases in thickness in a radially inward direction. In a tensioned configuration of a wire of the pair of wires, the ramped surface urges the tensioned wire toward a center of the pulley.

In a further aspect, a handle of a medical device includes: an actuator and a pulley. Actuation of the actuator causes rotation of the pulley. A pair of wires is coupled to the pulley by a crimp. The crimp extends across a gap in a portion of the pulley that is proximal when the actuator is unactuated.

The disclosed devices may include any of the following features, alone or in any combination. The gap is defined by three straight walls and an open outer side.

### BRIEF DESCRIPTION OF THE DRAWINGS

The accompanying drawings, which are incorporated in and constitute a part of this specification, illustrate aspects this disclosure and together with the description, and serve to explain the principles of the disclosure.

FIGS. 1A and 1B depict proximal and distal portions, respectively, of an exemplary medical device.

FIG. 2 shows a partial cutaway view of a handle of the medical device of FIGS. 1A and 1B, revealing aspects of a steering assembly.

FIG. 3A depicts an exploded view of the steering assembly of FIG. 2.

FIG. 3B illustrates an assembled view of the steering assembly of FIG. 3A.

FIGS. 4-6 depict views of a first pulley of the steering assembly of FIGS. 2-3B.

FIGS. 7-9 depict views of a second pulley of the steering assembly of FIGS. 2-3B.

FIG. 10 shows the first pulley of FIGS. 4-6 and the second pulley of FIGS. 7-9 in an assembled configuration.

### DETAILED DESCRIPTION

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values +/- 10% of a stated value. Unless otherwise stated, ranges disclosed herein include the end points of the ranges. The use of ordinals (e.g., first, second, third, fourth) herein is for convenient reference to different examples and does not imply any ordered or other type of relationship among identified elements. The ordinal applied to a given element is arbitrary, and any alternative ordinal may be used with the element.

A medical device, such as a scope device, may include a steering assembly for articulating a distal portion of a shaft of the medical device. The steering assembly may include pulleys. It will be appreciated that terms like “cam,” “wheel,” “disc,” or the like may be used in the art instead of the term “pulley,” and the term “pulley” as used herein encompasses structures with those (and other) similar terms. For example, the steering assembly may include two pulleys. One pulley may control articulation in one plane (e.g., left/right), and the other pulley may control articulation in another plane (e.g., up/down). Each pulley may be coupled to steering wires (e.g., to two steering wires). The steering wires may be coupled at their distal ends to an articulation section of the medical device.

As discussed herein, the steering wires may include multiple strands. The stranded wires may reduce friction and kinking compared to solid-core wires, improving both clinical performance and manufacturability. Furthermore, the stranded wires may not include a polytetrafluoroethylene (“PTFE”) coating, which may be present on some conventional, solid-core wires.

The steering wires may be coupled to the pulley via a crimp. The crimp may receive proximal ends of each of the two steering wires coupled to a given pulley. The crimp may be a hollow tube that, when exposed to a force, is compressed (crimped) to fixedly couple the wires to the crimp. Each pulley may, in some aspects, include a ramped surface that helps to inhibit the steering wires from disengaging (e.g., slipping off a side of) the pulley.

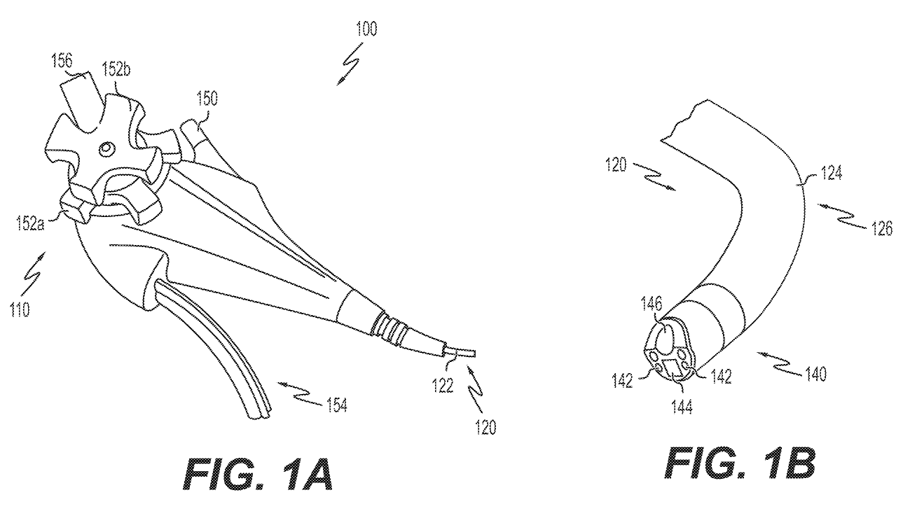

FIGS. 1A and 1B depict proximal and distal portions, respectively, of a medical device 100. In some examples, medical device 100 may be a cholangioscope. However, the medical device 100 may be another type of daughter scope, an endoscope, a ureteroscope, a duodenoscope, a gastroscope, an endoscopic ultrasonography (“EUS”) scope, a colonoscope, a bronchoscope, a laparoscope, an arthroscope, a cystoscope, an aspiration scope, a sheath, a catheter, a tome, or any type of accessory instrument having a shaft (e.g., a needle, a snare, a basket, a stent, a knife, a stapler, etc.). The aspects disclosed herein are not limited to use in a cholangioscope.

The medical device 100 may include a handle 110 and a shaft 120. The shaft 120 may be an elongated shaft. The shaft 120 may have a distal tip 140 coupled to or otherwise at a distal end of shaft 120. The shaft 120 may include a proximal portion 122 (FIG. 1A) and a distal portion 124 (FIG. 1B). The distal portion 124 of the shaft 120 and the distal tip 140 may be configured to be inserted into a body lumen of a subject. For example, the distal portion 124 of the shaft 120 and the distal tip 140 may be inserted into a working channel of a second medical device. The proximal portion 122 may remain outside of/external to the body lumen of the subject and the working channel of the second medical device. The distal portion 124 may include a steerable section 126, which may include an articulation joint or other structure (e.g., a multilumen extruded shaft or other bendable shaft) that is configured to deflect or otherwise articulate in order to steer the steerable section 126 and the distal tip 140.

The distal tip 140 may include one or more lighting elements 142, one or more imaging devices 144, and a distal opening 146 of a working channel of medical device 100. The one or more lighting elements 142, the one or more imaging devices 144, and the distal opening 146 may have any suitable arrangement. Alternatively or additionally, the distal tip 140 may include one or more end effectors (e.g., graspers, snares, staplers, clips, stents, forceps, suturing devices, baskets, etc.) for performing a medical procedure at a treatment site.

The handle 110 may include a port 150, which may provide access to the working channel of the medical device 100. An accessory instrument may be inserted into the port 150, advanced through the working channel, and out of the distal opening 146 of the distal tip 140. The handle 110 may also include one or more actuators 152a and 152b for controlling aspects of the shaft 120 and/or the distal tip 140. For example, one or more actuators 152a and 152b may include one or more steering knobs (or levers, sliders, etc.) for controlling articulation of the steerable section 126. For example, and as discussed in detail below, one or more wires may extend from the one or more actuators 152a and 152b to the steerable section 126, as discussed in detail below. Tensioning or de-tensioning of the wires may cause the steerable section 126 to deflect in one or more of an up/down or left/right direction. The handle 110 may include a lever 156, which may “lock” the actuators 152a, 152b, inhibiting them from moving (e.g., rotating). Thus, the lever 156 may serve to inhibit articulation of the steerable section 126 when the lever 156 is activated. The lever 156 may alternatively be another type of actuator (e.g., a knob, a button, a slider, etc.) Although one lever 156 is shown, the handle 110 may include one or more additional locking actuators (e.g., levers or knobs). Other actuators may control electronic elements of the distal tip 140 or deliver air, suction, and/or water.

An umbilicus 154 may extend from the handle 110. The umbilicus 154 may include one or more cables, tubes, or other conduits for conveying electrical signals, air, water, and/or suction to or from the handle 110, the shaft 120, and the distal tip 140. The umbilicus 154 may include one or more proximal connectors (not shown) for connecting to capital equipment, which may supply electrical controls, air, water, and/or suction.

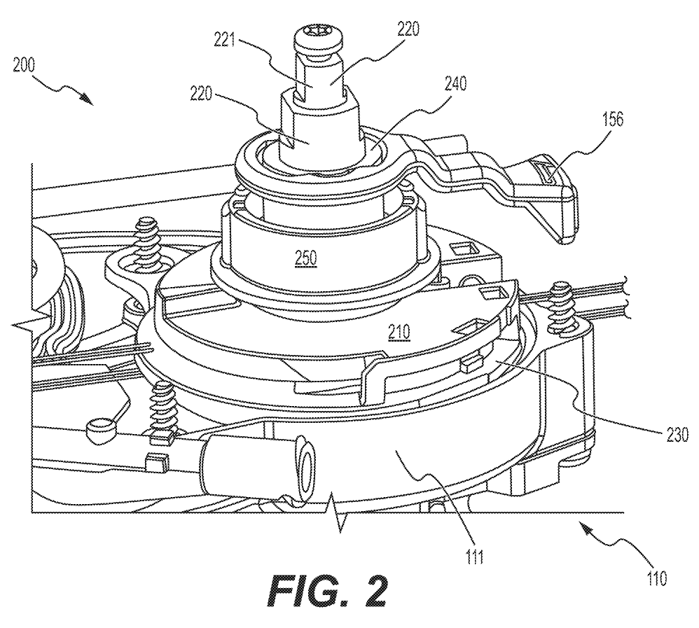

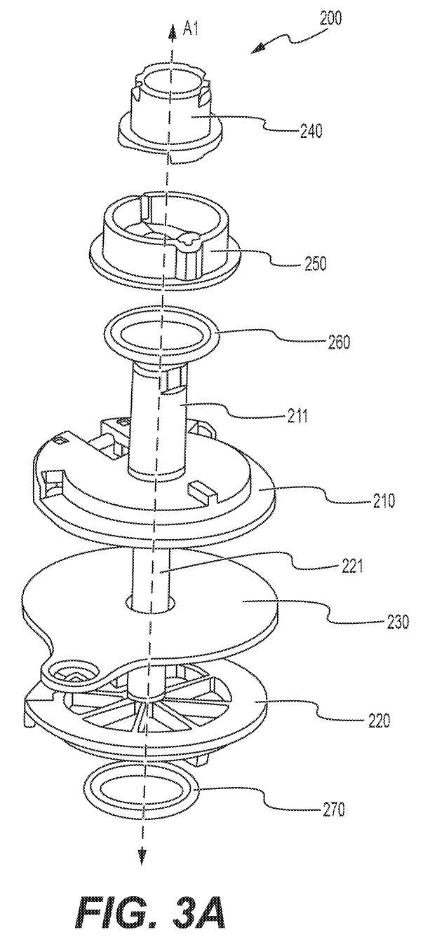

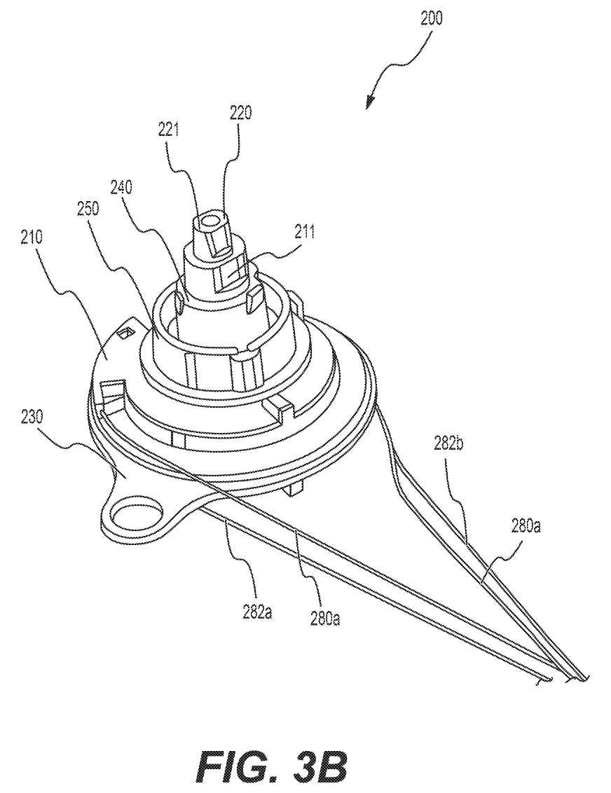

FIG. 2 shows a view of the handle 110 with a portion of a housing 111 of the handle 110 removed and with the actuators 152a, 152b removed to show internal components of a steering assembly 200 disposed at least partially within the housing of the handle 110. FIG. 3A illustrates an exploded view of the steering assembly 200, while FIG. 3B shows the steering assembly 200 in an assembled configuration.

The steering assembly 200 may be configured to control movement of the steerable section 126 of the shaft 120, in conjunction with the actuators 152a, 152b and the lever 156.

The steering assembly 200 may include a first pulley 210 and a second pulley 220. The first pulley 210 and the second pulley 220 may be arranged in a stacked configuration. A plate 230 may be positioned between the first pulley 210 and the second pulley 220. The plate 230 may rotationally isolate the first pulley 210 and the second pulley 220 from one another, helping to facilitate independent rotation of the first pulley 210 and the second pulley 220. In other words, the first pulley 210 and the second pulley 220 may each independently rotate without the other rotating. The steering assembly 200 may further include a cam 240 and a sleeve 250. In some cases, as discussed below, the cam 240 and the sleeve 250 may cooperate with the lever 156 to selectively inhibit rotation of the first pulley 210 and the second pulley 220.

The first pulley 210 may be operatively connected to the first actuator 152a by the axle 211. Thus, rotation of the actuator 152a may cause corresponding rotation of the first pulley 210. A proximal portion of a first wire 280a and a proximal portion of a second wire 280b may be coupled to the first pulley 210, as discussed in further detail below. Wires 280a, 280bmay form a first pair of wires. A distal portion of the first wire 280aand a distal portion of the second wire 280b may be coupled to the steerable section 126. As the first pulley 210 rotates, one of the wires 280a, 280bmay be moved proximally and the other of the wires 280a, 280bmay be moved distally. Thus, the rotation of the first pulley 210 may articulate the steerable section 126 along a first plane (e.g., an up/down or a left/right plane).

Similarly, the second pulley 220 may be operatively connected to the second actuator 152b by an axle 221. Thus, rotation of the actuator 152bmay cause corresponding rotation of the second pulley 220. A proximal portion of a first wire 282aand a proximal portion of a second wire 282b may be coupled to the second pulley 220, as discussed in further detail below. Wires 282a, 282bmay form a second pair of wires. A distal portion of the first wire 282a and a distal portion of the second wire 282b may be coupled to the steerable section 126. As the second pulley 220 rotates, one of the first wire 282a or the second wire 282b may be moved proximally and the other of the first wire 282a or the second wire 282b may be moved distally. Thus, the rotation of the second pulley 220 may articulate the steerable section 126 in a second plane, which may be perpendicular to the first plane. For example, the first pulley 210 may cause articulation in the up/down direction, and the second pulley 220 may cause articulation in the left/right direction, or vice versa. This arrangement may allow for independent control of different directions of movement of the steerable section 126.

Each of the wires 280a, 280b, 282a, 282b may be a multi-strand wire. Compared with conventional solid-core steering wires, multi-strand wires 280 280a, 280b, 282a, 282b reduce friction and kinking, improving both clinical performance and manufacturability. This may allow the wires 280a, 280b, 282a, 282bto lack a PTFE coating, which may be used on conventional solid-core wires. It will be appreciated that the term “wire” is not limited to single-strand wires and includes multi-strand wires, as discussed above, along with cables. The wires 280a, 280b, 282a, 282b may be made from metallic material or from non-metallic materials.

The components of the steering assembly 200 may be arranged in a compact configuration, at least partially within a housing of the handle 110. The axles 211, 221 may be rotatable about an axis A1 (FIG. 3A). Axis A1 may define an axial direction. An axially outer direction (or a first axial direction) may be a direction toward the actuators 152a, 152band may be a generally upward direction in FIGS. 2-3B. An axially inner direction (or second axial direction) may be a direction away from the actuators 152a, 152b and may be a generally downward direction in FIGS. 2-3B. The first pulley 210 may be an axially outer pully, and the second pulley 220 may be an axially inner pulley. A radial direction as described herein may be relative to axis A1, with a radially inner direction being a direction toward axis A1 and a radially outer direction being a direction away from axis A1.

The cam 240 and the sleeve 250 may be axially outward of the first pulley 210 and the second pulley 220. The cam 240 may be received radially inwardly of and within the sleeve 250. In some aspects, the cam 240 and the sleeve 250 may be at least partially outside of the housing of the handle 110 and partially inside of the housing of the handle 110. The first pulley 210 and the second pulley 220 may be within (inside of) the housing of the handle 110. The cam 240 may be keyed to the lever 156, such that rotation of the lever 156 causes corresponding rotation of the cam 240. The cam 240 and the sleeve 250 may include surfaces that are configured to interact so that, upon rotation of the cam 240, the sleeve 250 moves axially inward. In cooperation with a first O-ring 260, positioned axially outward of and adjacent to the first pulley 210, and a second O-ring 270, positioned axially inward of and adjacent to the second pulley 220, movement of the sleeve 250 in an axially inward direction may inhibit the first pulley 210 and the second pulley 220 from rotating. Thus, the lever 156, the cam 240, and the sleeve 250 may cooperate to “lock” the steering assembly 200. The locking mechanism described above is merely exemplary, and any other suitable locking mechanisms may be used.

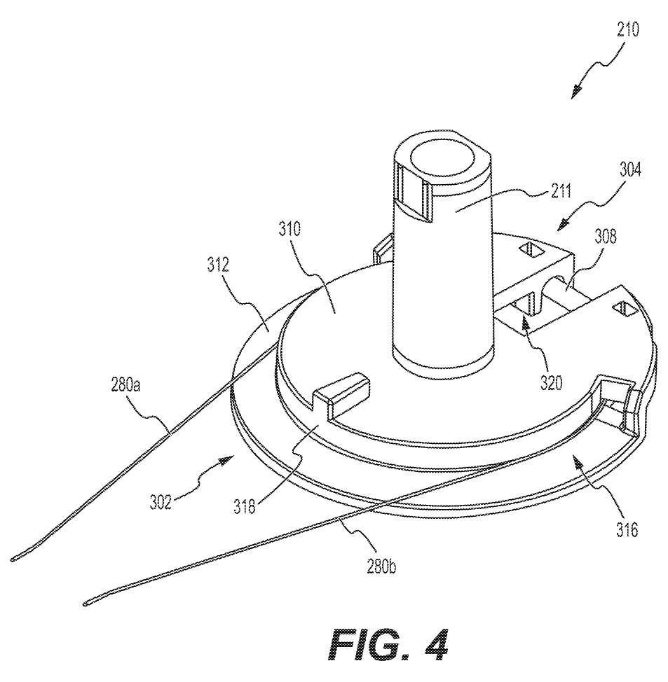

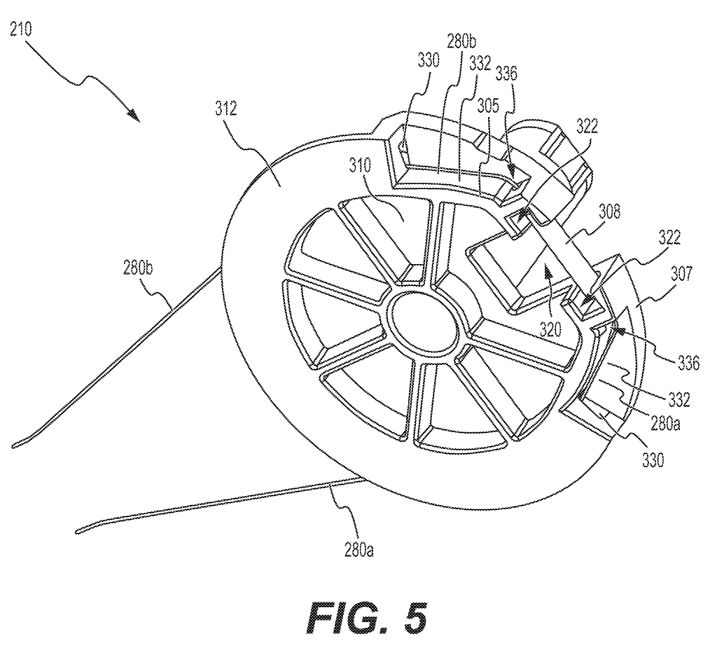

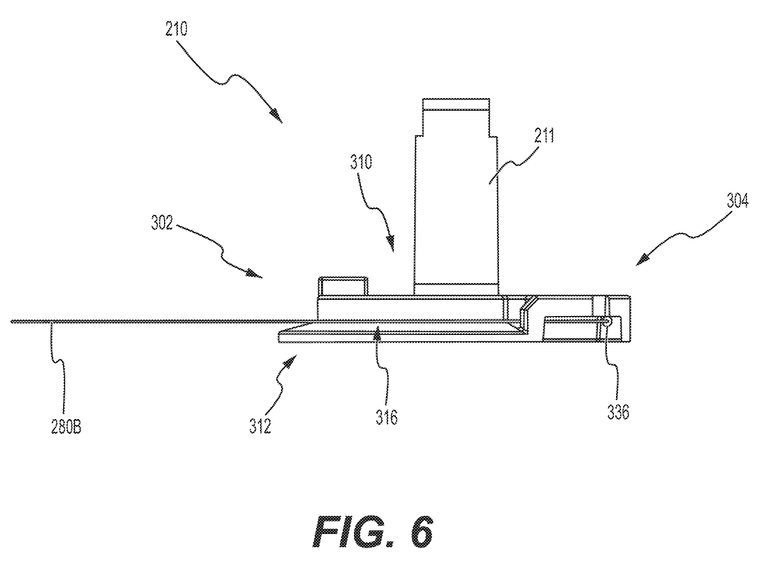

FIG. 4 illustrates a perspective view of an axially outer side of the first pulley 210. FIG. 5 shows a perspective view of an axially inner side of the first pulley 210. FIG. 6 depicts a side view of the first pulley 210. As discussed above, the axial direction is defined by the axis A1, which is coaxial with the axle 211 and is an axis of rotation of the first pulley 210. The first pulley 210 may include a first portion 302 and a second portion 304. The first portion 302 may be a distal portion of first pulley 210 when the pulley 210 is in a neutral (unactuated) configuration (e.g., when first actuator 154a is unactuated). The second portion 304 may be a proximal portion of the first pulley 210, when the first pulley 210 is in a neutral configuration. The axle 211 may be keyed to or otherwise rotationally fixedly coupled to the first actuator 152a, as discussed above. In some aspects, the first portion 302, the second portion 304, and the axle 211 may be integrally formed from a single piece of material. The pulley 210 may have an overall approximately round perimeter. A crimp 308 may be used to couple the wires 280a, 280bto the first pulley 210. The crimp 308 may have a length of 0.4 in, an outer diameter of 0.078 in and an internal diameter of 0.036 in. In another example, the crimp 308 may have a length between 0.2 to 0.3 inches, 0.3 to 0.4 inches, or 0.4 to 0.5 inches. The outer diameter of the crimp 308 may be between 0.05 to 0.06 inches, 0.06 to 0.07 inches, or 0.07 to 0.08 inches. The inner diameter of the crimp 308 may be between 0.01 to 0.02 inches, 0.02 to 0.03 inches, or 0.03 to 0.04 inches.

The first portion 302 may comprise a radially inner portion 310 (e.g., a central hub) and a radially outer portion 312. The outer portion 312 may have a flange shape around the inner portion 310. The outer portion 312 may be recessed relative to inner portion 310. In other words, the outer portion 312 may extend only around an axially inner portion of the inner portion 310.

The outer portion 312 may include a ramped surface 316. Ramped surface 316 may be angled axially outward moving in a radially inward direction. In other words, moving radially inward from an outer perimeter of the outer portion 312, the ramped surface 316 may slope or incline axially outward. Said another way, the ramped surface 316 may slope upward in FIG. 6, moving from an outer perimeter of the outer portion 312, toward the inner portion 310 (toward a center of pulley 210). Thus, the outer portion 312 may have a greater thickness closer to the inner portion 310 than near an outer perimeter of the outer portion 312. In other words, the outer portion 312 may have an increasing thickness moving in a radially inward direction, such that more radially inward portions of the outer portion 312 are thicker. The ramped surface 316 may extend only partially around the first pulley 210—only around the first portion 302.

The ramped surface 316 may inhibit the first wire 280aand the second wire 280b from slipping off of the first pulley 210. The wires 280a, 280bmay be pushed toward an outer wall 318 of the inner portion 310 when the respective first wire 280a or second wire 280b is in tension. When the wires 280a, 280 bis in tension from articulation, the wires 280a, 280b presses into the ramped surface 416, which in turn drives the wire up the ramp and into the inner portion 310. In other words, the ramped surface 316 may urge the wires 280a, 280b toward a center of the pulley 210 when the wires 280a, 280bis tensioned.

Pulleys lacking a ramped surface 316 may require expensive components and tight tolerances to mitigate slipping and jumping. In contrast, the ramped surface 316 guides the wires 280a, 280b securely within the outer diameter of the first pulley 210, inhibiting the wires 280a, 280b slipping under or over the system and ensuring reliable articulation throughout a clinical procedure. This design achieves reliable wire positioning while allowing for the simplification of mold tooling. Tooling simplifications allow for longer life, wider tolerancing, and ultimately a lower cost part.

The second portion 304 may have a radially inner portion 305 and a radially outer portion 307 (shown particularly in FIG. 5). Whereas the inner portion 310 of the first portion 302 may extend further in an axial outer direction, the inner portion 305 of the second portion 304 may extend further axially inwardly as compared to the outer portion 307 of the second portion 304. The outer portion 307 of the second portion 304 may be approximately axially level with the inner portion 310 of the first portion 302. The inner portion 305 of the second portion 304 may be approximately axially level with the outer portion 312 of the first portion 302.

The second portion 304 of the first pulley 210 may include a gap 320 that may extend from an outer perimeter of the second portion 304 toward the axle 211. The gap 320 may extend through the outer portion 307 and through a portion of the inner portion 305. The gap 320 may be positioned on the proximalmost side of the first pulley 210 when in a neutral (unactuated) configuration. For example, the gap 320 may be centered on an axis extending in a proximal/distal direction of the handle 110.

The gap 320 in the first pulley 210 may be defined by three straight walls and an open radially outer side. The gap 320 may extend from an axially outer side of the pulley 210 to an axially inner side of the first pulley 210, being open on all sides except for the three straight walls. The gap 320 may extend from an outer perimeter of the pulley 210 toward the axle 211 but may terminate radially outwardly of the axle 211. The second portion 304 may include notches 322 positioned on either lateral side of the gap 320 near the outer perimeter of the second portion 304 of the first pulley 210.

The crimp 308 may be received within the notches 322. The first crimp may be formed of a metal, such as copper (or another suitable material). The crimp 308 may define an inner lumen for receiving the first wire 280a and the second wire 280b. As discussed below, proximal ends of the wires 280a, 280b may be inserted into the lumen of the crimp 308, before or after the crimp 308 is inserted into the notches 322. The crimp 308 may be compressed (crimped) to secure the proximal ends of the wires 280a, 280b within the crimp 308, thereby coupling the wires 280a, 280b to the first pulley 210. The crimp 308 may be secured within the notches 322 (e.g., fixedly coupled to the notches 322) by a friction fit, by adhesive, by tension, or by other mechanisms. A size (length and outer diameter) of the crimp 308 may be chosen so that crimp 308 is securely held within notches 322.

The crimp 308 may be a substantially straight tube before it is crimped. The crimp 308 may form a portion of an outer perimeter of the first pulley 210. For example, crimp 308 may be a radially outermost structure of the first pulley 210 along a portion of the first pulley 210 (a portion having the gap 320). The crimp 308 may extend from one surface of the second portion 304 to another surface of the second portion 304, with a gap/space between the surfaces. A longitudinal axis extending along crimp 308 (e.g., through a central lumen of crimp 308 before it is crimped) may be substantially perpendicular to rotational axis A1 and axle 211.

As mentioned above, distal portions of the wires 280a, 280b may be coupled to steerable section 126. The wires 280a, 280bmay extend through shaft 120, to handle 110. A proximal portion of wires 280a, 280bmay extend along first portion 302 (e.g., along ramped surface 316 of outer portion 312), on opposite sides of first pulley 210. Each of wires 280a, 280b may extend through a respective opening 330 on opposite sides of the first pulley, between the first portion 302 and the second portion 304. Along the first portion 302, the wires 280a, 280b may be disposed on an axially outer side (e.g., an axially outer surface, which may be the ramped surface 316). Along the second portion 304, the wires 280a, 280b may extend on an axially inner side of the second portion 304. The openings 330 may allow passage of the first wire 280a and the second wire 280b between these opposite axial sides of the first pulley 210. An outer surface 332 of the inner portion 305 of the second portion 304 may provide a path along which the first wire 280a and the second wire 280b may travel along the second portion 304, toward the crimp 308. The outer surface 332 may be on an axially inner surface of the second portion 304, and may be radially recessed relative to an outer perimeter of the second portion 304.

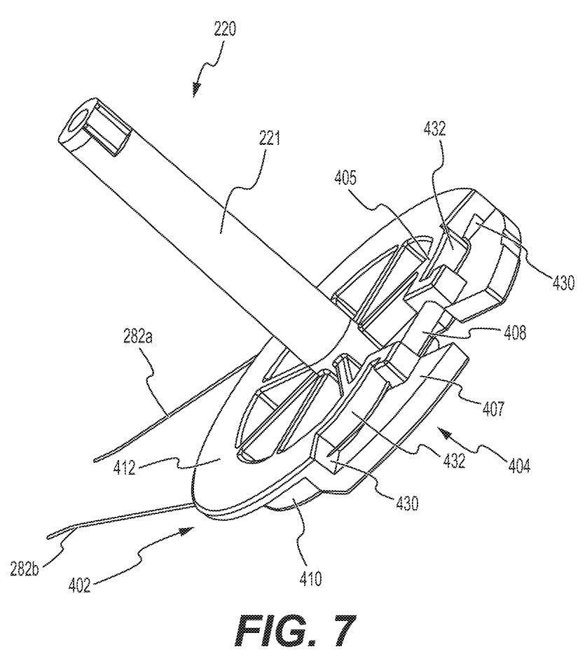

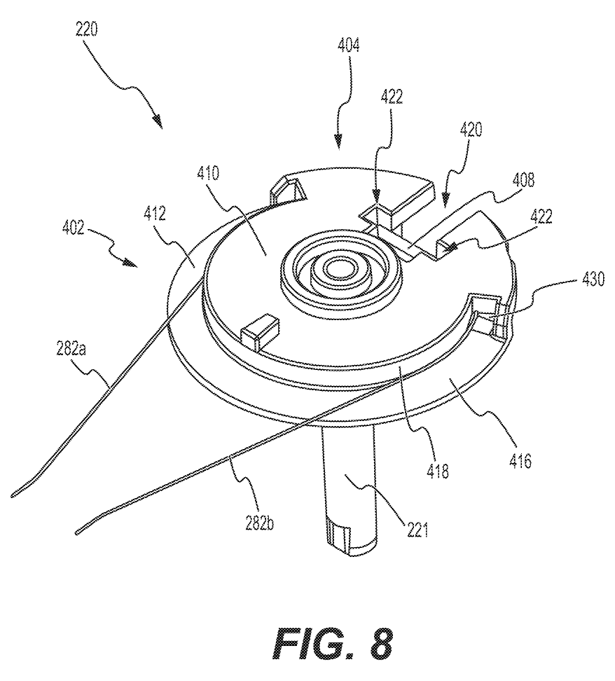

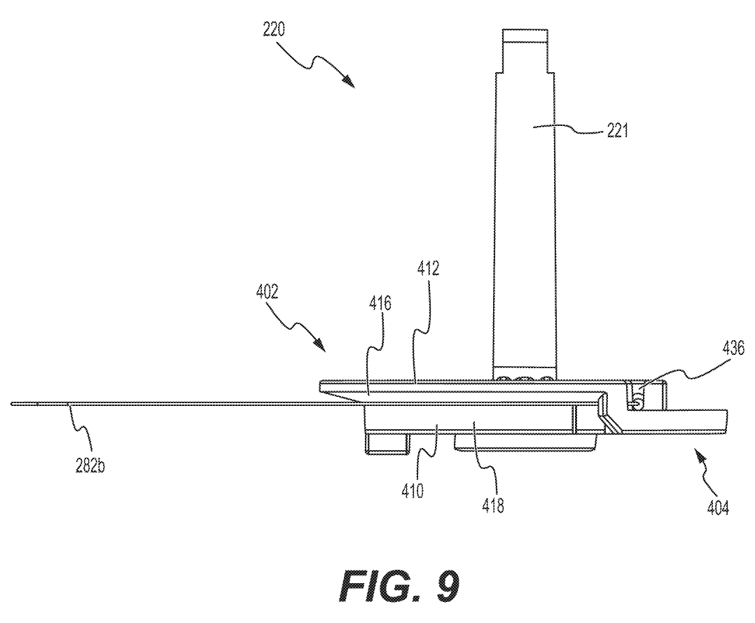

FIG. 7 illustrates a perspective view of an axially outer side of the second pulley 220, wherein the axially outer side is facing the actuators 152a, 152b, the plate 230, and the first pulley 210. FIG. 8 shows a perspective view of the second pulley 220 from an axially inner side, wherein the axially inner side is a side facing away from the pulley 210 and the plate 230. FIG. 9 illustrates a side view of the second pulley 220. As discussed above, direction is defined by the axis A1, which is coaxial with the axle 221 and is an axis of rotation of the second pulley 220.

The second pulley 220 may be substantially similar to the first pulley 210. Differences between the second pulley 220 and the first pulley 210 are noted below. For example, certain directions may be reversed with respect to the pulleys 210, 220. This is because the respective wires 280a, 280band 282a, 282b are received on sides of the pulleys 210, 220 that face away from one another, to provide for increased spacing between the wires 280a, 280b (a first wire pair) and wires 282a, 282b (a second wire pair).

The second pulley 220 may include a first portion 402 and a second portion 404. The first portion 402 may be a distal portion of the second pulley 220 when the second pulley 220 is in a neutral (unarticulated) configuration. The second portion 404 may be a proximal portion of the second pulley 220, when the second pulley 220 is in a neutral configuration. The axle 221 may be keyed to or otherwise rotationally fixedly coupled to the second actuator 152b, as discussed above. In some aspects, the first portion 402, the second portion 404, and the axle 221 may be integrally formed from a single piece of material. The pulley 220 may have an overall approximately round perimeter. A crimp 408 may be used to couple the wires 282a, 282bto the second pulley 220. The crimp 408 may have a length of 0.4 in, an outer diameter of 0.078 in and an internal diameter of 0.036 in. In another example, the crimp 408 may have a length between 0.2 to 0.3 inches, 0.3 to 0.4 inches, or 0.4 to 0.5 inches. The outer diameter of the crimp 408 may be between 0.05 to 0.06 inches, 0.06 to 0.07 inches, or 0.07 to 0.08 inches. The inner diameter of the crimp 408 may be between 0.01 to 0.02 inches, 0.02 to 0.03 inches, or 0.03 to 0.04 inches.

The first portion 402 may comprise a radially inner portion 410 (e.g., a central hub) and a radially outer portion 412. The outer portion 412 may have a flange shape around the inner portion 410. The outer portion 412 may extend only around an axially outer portion of the inner portion 410. The inner portion 410 may extend further axially inward than the outer portion 412 does.

The outer portion 412 may include a ramped surface 416. The ramped surface 416 may be angled axially inward moving in a radially inward direction. In other words, moving radially inward from an outer perimeter of the outer portion 412, the ramped surface 416 may slope or incline axially inward. Said another way, the ramped surface 416 may slope downward in FIG. 6, moving from an outer perimeter of the outer portion 412, toward the inner portion 410 (toward a center of pulley 220). Thus, the outer portion 412 may have a greater thickness closer to inner portion 410 than near an outer perimeter of the outer portion 412. In other words, the outer portion 412 may have an increasing thickness moving in a radially inward direction, such that more radially inward portions of the outer portion 412 are thicker. The ramped surface 416 may extend only partially around the pulley 220—only around the first portion 402.

The ramped surface 416 may inhibit the first wire 282aand the second wire 282b from slipping off of the second pulley 220. The first wire 282aor the second wire 282bmay be pushed toward an outer wall 418 of the inner portion 410 when the respective first wire 282a or second wire 282b is in tension. When the wire 282a or 282bis in tension from articulation, the wire 282a or 282b presses into the ramped surface 416, which in turn drives the wire into the inner portion 410. In other words, the ramped surface 416 may urge wire 282aor 282b toward a center of pulley 220 when the wire 282a or 282b is tensioned.

Pulleys lacking a ramped surface 416 may require expensive components and tight tolerances to mitigate slipping and jumping. In contrast, ramped surface 416 guides the wires 282a, 282b securely within the outer diameter of the second pulley 220, inhibiting the wires 282a, 282b slipping under or over the system and ensuring reliable articulation throughout a clinical procedure. This design achieves reliable wire positioning while allowing for the simplification of mold tooling. Tooling simplifications allow for longer life, wider tolerancing, and ultimately a lower cost part.

The second portion 404 may have a radially inner portion 405 and a radially outer portion 407 (shown particularly in FIG. 5). Whereas the inner portion 410 of the first portion 402 may extend further in an axial inner direction, the inner portion 405 of the second portion 404 may extend further axially outwardly as compared to outer portion 407 of the second portion 404. The outer portion 407 of the second portion 404 may be approximately axially level with the inner portion 410 of the first portion 402. The inner portion 405 of the second portion 404 may be approximately axially level with the outer portion 412 of the first portion 402.

The gap 420 in the second pulley 220 may be defined by three straight walls and an open radially outer side. The gap 420 may extend from an axially outer side of the pulley 220 to an axially inner side of the second pulley 220, being open on all sides except for the three straight walls. The gap 420 may extend from an outer perimeter of the pulley 220 toward the axle 211 but may terminate radially outwardly of the axle 211. The second portion 404 may include the notches 422 positioned on either lateral side of the gap 420 near the outer perimeter of the second pulley 220/the second portion 404. As compared with the notches 322 of the first pulley 210, the notches 422 of the second pulley 220 may be recessed further radially inward, closer to the axle 221.

The crimp 408 may be received within the notches 422. As discussed in further detail below, the crimp 408 may be located closer to axle 221/axis A1 than the crimp 308 is to axle 211/axis A1. The first crimp may be formed of a metal, such as copper (or another suitable material). The crimp 408 may define an inner lumen for receiving the first wire 282a and the second wire 282b. As discussed below, proximal ends of the wires 282a, 282b may be inserted into the lumen of the crimp 408, before or after the crimp 408 is inserted into the notches 422. The crimp 408 may be compressed (crimped) to secure the proximal ends of the wires 282a, 282b within the crimp 408, thereby coupling the wires 282a, 282b to the second pulley 220. The crimp 408 may be secured within the notches 422 (e.g., fixedly coupled to the notches 422) by a friction fit, by adhesive, by tension, or by other mechanisms.

The crimp 408 may be a substantially straight tube before it is crimped. The crimp 408 may form a portion of an outer perimeter of the second pulley 220. For example, crimp 408 may be a radially outermost structure of the second pulley 220 along a portion of the second pulley 220 (a portion having the gap 420). The crimp 408 may extend from one surface of the second portion 404 to another surface of the second portion 404, with a gap/space between the surfaces. A longitudinal axis extending along crimp 408 (e.g., through a central lumen of crimp 408 before it is crimped) may be substantially perpendicular to rotational axis A1 and axle 221.

As mentioned above, distal portions of the wires 282a, 282b may be coupled to steerable section 126. The wires 282a, 282b may extend through shaft 120, to handle 110. A proximal portion of wires 282a, 282b may extend along first portion 402 (e.g., along ramped surface 416 of outer portion 412), on opposite sides of second pulley 220. Each of wires 282a, 282b may extend through a respective opening 430 on opposite sides of the first pulley, between the first portion 402 and the second portion 404. Along the first portion 402, the wires 282a, 282b may be disposed on an axially inner side (e.g., an axially inner surface, which may be the ramped surface 416). Along the second portion 404, the wires 282a, 282b may extend on an axially outer side of the second portion 404. The openings 430 may allow passage of the first wire 282a and the second wire 282b between these opposite axial sides of the second pulley 220. An outer surface 432 of the second portion 404 may provide a path along which the first wire 282a and the second wire 282b may travel along the second portion 404, toward the crimp 408. The outer surface 432 may be on an axially outer side of the second portion 404, and may be radially recessed relative to an outer perimeter of the second portion 304.

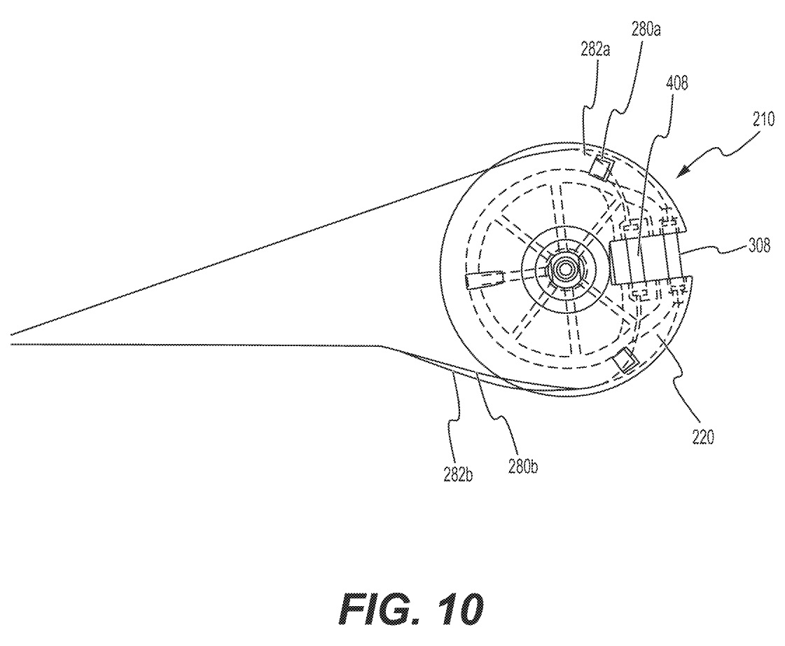

FIG. 10 illustrates a side view showing the arrangement of the crimps 308 and 408 when the pulleys are assembled with axle 221 inside of axle 211. As shown in FIG. 10, crimp 308 may be positioned at a more radially outward location than crimp 408. Thus, longitudinal axes of the crimps 308, 408 may be substantially parallel to one another but not coaxial with one another. These different radial locations may allow for crimp 308 and crimp 408 to be crimped in a same pass, by a same machine, thereby streamlining manufacturing.

As discussed above, the crimp 308 and the second crimp 408 may each be made from a malleable metal material, such as copper. The malleable nature of the material may allow the crimps to deform, thereby closing onto and securing the respective wires 280a, 280b or 282a, 282b.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of these embodiments can be combined to achieve a same or similar result as one or more of the disclosed embodiments. Accordingly, the invention is not to be considered as limited by the foregoing description.

We claim:

1. A handle of a medical device, the handle comprising:
an actuator;
a pulley, wherein actuation of the actuator causes rotation of the pulley; and
a pair of wires coupled to the pulley by a crimp, wherein a longitudinal axis of the crimp is approximately perpendicular to an axis of rotation of the pulley.

2. The handle of claim 1, wherein the actuator is a first actuator, the pulley is a first pulley, the pair of wires is a first pair of wires, the crimp is a first crimp, and the longitudinal axis is a first longitudinal axis, the handle further comprising:
a second actuator;
a second pulley, wherein actuation of the second actuator causes rotation of the second pulley; and
a second pair of wires coupled to the second pulley by a second crimp, wherein a second longitudinal axis of the second crimp is approximately perpendicular to the axis of rotation.

3. The handle of claim 2, wherein the first longitudinal axis and the second longitudinal axis are approximately parallel to one another and are not coaxial.

4. The handle of claim 1, wherein each wire of the pair of wires is a multi-strand wire.

5. The handle of claim 1, wherein the pulley includes a ramped surface that slopes toward a center of the pulley.

6. The handle of claim 5, wherein, in a tensioned configuration of a wire of the pair of wires, the ramped surface urges the tensioned wire toward the center of the pulley.

7. The handle of claim 5, wherein the pulley includes a radially inner portion and a radially outer portion, wherein the radially inner portion is closer to a rotational axis of the pulley, and wherein the ramped surface slopes in a direction toward the radially inner portion.

8. The handle of claim 7, wherein the radially outer portion has a flange shape.

9. The handle of claim 5, wherein the ramped surface extends only partially around the pulley.

10. The handle of claim 1, wherein the pulley includes a gap extending from an outer perimeter of the pulley toward an axle of the pulley.

11. The handle of claim 10, wherein the crimp extends across the gap.

12. The handle of claim 11, wherein the pulley includes a recess on either side of the gap for receiving the crimp.

13. The handle of claim 10, wherein the gap is defined by three straight walls and an open outer side.

14. The handle of claim 10 wherein the pulley includes openings such that the wires are on a first axial side of the pulley on a proximal portion of the pulley and a second axial side of the pulley on a distal portion of the pulley.

15. The handle of claim 14, wherein the gap is on the proximal portion of the pulley.

16. A handle of a medical device, the handle comprising:
an actuator;
a pulley, wherein actuation of the actuator causes rotation of the pulley; and
a pair of wires coupled to the pulley, wherein the pulley includes a radially inner portion and a radially outer portion, wherein the radially outer portion includes a ramped surface that is inclined toward the radially inner portion.

17. The handle of claim 16, wherein the radially outer portion increases in thickness in a radially inward direction.

18. The handle of claim 16, wherein, in a tensioned configuration of a wire of the pair of wires, the ramped surface urges the tensioned wire toward a center of the pulley.

19. A handle of a medical device, the handle comprising:
an actuator;
a pulley, wherein actuation of the actuator causes rotation of the pulley; and
a pair of wires coupled to the pulley by a crimp, wherein the crimp extends across a gap in a portion of the pulley that is proximal when the actuator is unactuated.

20. The handle of claim 19, wherein the gap is defined by three straight walls and an open outer side.
